(19) United States
(12) Patent Application Publication
(10) Pub. No.: US 2018/0088059 A1
(43) Pub. Date: Mar. 29, 2018

(54) FLUORESCENT DENDRITIC TAGS

(71) Applicant: Michael Kozicki, Phoenix, AZ (US)

(72) Inventor: Michael Kozicki, Phoenix, AZ (US)

(21) Appl. No.: 15/711,643

(22) Filed: Sep. 21, 2017

Related U.S. Application Data
(60) Provisional application No. 62/399,882, filed on Sep. 26, 2016.

(51) Int. Cl. G01N 21/91; G01N 21/64; A61K 47/64; C12N 5/0784

(52) U.S. Cl. CPC G01N 21/91; G01N 21/6428; A61K 2039/5154; C12N 5/0639; A61K 47/641

(57) ABSTRACT

A method of detecting a covert marking including applying a fluorescent ink to a dendritic structure to generate an ink-coated dendrite and imaging the ink-coated dendrite with a device to visualize the ink-coated dendrite. A method of detecting a covert marking on an object including applying UV light to an object including a dendritic structure and illuminating the dendritic structure with the UV light. A dendritic structure including a unique metallic structure at least partially coated with a fluorescent ink that is not visible to the naked eye in ambient light and is visible to the naked eye when exposed to UV light.

### CROSS-REFERENCE TO RELATED APPLICATIONS

This application is a non-provisional of and claims the benefit of U.S. Provisional Application No. 62/399,882, filed on Sep. 26, 2016, the entire contents of which are incorporated herein by reference.

### BACKGROUND OF THE INVENTION

A dendritic structure is a structure that develops with a typical multi-branching, tree-like form. Dendritic patterns are very common in nature and are illustrated by diverse phenomena such as snowflake formation and lightning. Dendritic crystallization forms a natural fractal pattern. A fractal is generally defined as a rough or fragmented geometric shape that can be subdivided into parts, each of which is (at least stochastically) a reduced-size copy of the whole, a property called self-similarity. This self-similarity leads to a fine structure at arbitrarily small scales. Because they appear similar (but not identical) at all levels of magnification, fractals are often considered to be infinitely complex. In practice, however, the finest observable levels of structure will be limited by physical and/or chemical constraints.

In general, a dendritic metal structure can be formed by the electrodeposition of ions on or in an ion conductor. There are several viable options for the composition of the ion conductor, which can exist as a liquid, solid, or gel. Metals such as silver and copper are particularly appropriate as they are highly mobile in a variety of materials and are readily reduced and oxidized, which makes the electrochemical aspects of the process relatively straightforward.

The dendritic structures disclosed herein can be used as identification tags for a variety of commercial transactions and security applications. Due to their complex nature, dendritic structures are unique and therefore function as “fingerprints,” enabling unique tagging and later identification of a wide variety of articles. Use of dendritic structures for identification and authentication applications entails robust analysis and recognition of the structures. Accordingly, the disclosure features methods and systems for detecting or visualizing dendritic structures.

### SUMMARY OF THE INVENTION

In one embodiment, the invention provides a method of detecting a covert marking. The method comprises applying a fluorescent ink to a dendritic structure to generate an ink-coated dendrite and imaging the ink-coated dendrite with a device to visualize the ink-coated dendrite.

In another embodiment, the invention provides a method of detecting a covert marking on an object. The method comprises applying UV light to an object including a dendritic structure and illuminating the dendritic structure with the UV light.

In yet another embodiment, the invention provides a dendritic structure comprising a unique metallic structure at least partially coated with a fluorescent ink that is not visible to the naked eye in ambient light and is visible to the naked eye when exposed to UV light.

Other aspects of the invention will become apparent by consideration of the detailed description and accompanying drawings.

### BRIEF DESCRIPTION OF THE DRAWINGS

The patent or application tile contains at least one drawing executed in color. Copies of this patent or patent application publication with color drawing(s) will be provided by the Office upon request and payment of the necessary tee.

FIG. 1A illustrates an optical micrograph of 10 nm thick Ag dendrite grown on Ag—Ge₃Se₇ solid electrolyte on a Si wafer (gray background). This image is a view of an about 2 mm wide portion of the dendritic growth, showing no discernible features.

FIG. 1B illustrates an optical micrograph of 10 nm thick Ag dendrite grown on Ag—Ge₃Se₇ solid electrolyte on a Si wafer (gray background). This image is a 10× magnification view of the electrodeposit in FIG. 1A, revealing a faint dendritic pattern.

FIG. 1C illustrates an optical micrograph of 10 nm thick Ag dendrite grown on Ag—Ge₃Se₇ solid electrolyte on a Si wafer (gray background). This image illustrates a contrast-adjusted version of FIG. 1B, highlighting position and shape of the electrodeposit.

FIG. 2 is a schematic of a dendrite growth chip, including multiple common cathode pillars aligned with common circular anodes. An isolation layer prevents electrodeposition between the cathode and anode layers.

FIG. 3A illustrates a layout of dendrite growth devices with a 200 μm diameter anode (560/array). Contact to the common cathodes is via probe pad holes at the top left and bottom right corners of each array. Anode contacts are at opposite corners.

FIG. 3B illustrates a layout of dendrite growth devices with 800 μm diameter anode (60/array). Contact to the common cathodes is via probe pad holes at the top left and bottom right corners of each array. Anode contacts are at opposite corners.

FIG. 3C illustrates a layout of dendrite growth devices with 2400 μm diameter anode (9/array). Contact to the common cathodes is via probe pad holes at the top left and bottom right corners of each array. Anode contacts are at opposite corners.

FIG. 4 is a photograph of a Type B (800 μm diameter anode) array.

FIG. 5 is a micrograph of a Type B (800 μm diameter anode) array showing simultaneous dendrite growth at all locations.

FIG. 6A is a micrograph of Type A (200 μm diameter anode) devices following dendrite growth. Growth is at 2 V with a fractal dimension of 1.20.

FIG. 6B is a micrograph of Type A (200 μm diameter anode) devices following dendrite growth. Growth is at 1 V with a fractal dimension of 1.43.

FIG. 7A is a micrograph of a Type A (200 μm diameter) device following dendrite growth. Growth is at 2 V.

FIG. 7B is a micrograph of a Type B (800 μm diameter) device following dendrite growth. Growth is at 2 V.

FIG. 8A illustrates a light path of a microscope (bright field).

FIG. 8B illustrates a light path of a microscope (dark field).

FIG. 9 illustrates bright field (left) and dark field (right) images for dendrites in a Type A device grown at 1 V (top), in a Type A device grown at 2 V (middle), and at a Type B device grown at 2 V (bottom).

FIG. 10 illustrates statistical results for fractal dimension (FD) and pattern size of 23 dendritic patterns formed simultaneously on a Type B die.

FIG. 11A illustrates a light field image of a dendrite grown at 2 V on a Type B die.

FIG. 11B illustrates a dark field image of a dendrite grown at 2 V on a Type B die.

FIG. 12 illustrates a fluorescence microscope image of a fluorescent ink-coated dendrite grown at 2 V on a Type B die.

### DETAILED DESCRIPTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways.

Covert markings are completely invisible to the human eye. Covert markings require a reading device to detect or visualize the marking. Objects can be tagged with a covert marking and later checked with a reading device to assess authenticity. Dendritic structures according to some embodiments of the invention are employed for covert marking of objects.

It is also noted that while particle-based inks were employed in the exemplary embodiments described below, dye-based inks can also work.

Fluorescent inks and toners may be used as an authenticating feature in the document security industry. Secure documents, for example documents that are difficult to forge, may be created using inks or toners that include fluorescent agents either alone or in combination with ordinary inks and/or pigments. Features printed using fluorescent inks or toners are usually invisible under visible light, due to the colorless nature of the security inks or due to masking by other colorants in the document. Under ultraviolet illumination, however, the fluorescent features of the document are revealed in the form of a bright emission by the fluorescent dyes in the visible spectrum. For example, certain bank notes utilize visible features, such as holographic patches, microprinting, and microtextures to conceal additional fluorescent threads and/or multi-colored emblems embedded in the bank note, which are only revealed under specific light frequencies. These features provide an increased level of security against counterfeiters by making the copying process of such a document more difficult.

The term “fluorescent dye” refers to a fluorescent material that is soluble, such as an organic molecule in a vehicle, and easily makes homogeneous printing compositions.

The term “fluorescent pigment” refers to a fluorescent material that is insoluble in a vehicle and generally requires substituted uniform dispersion in the vehicle to use it. In most cases, the only medium available that may dissolve fluorescent pigments is a strong acid, such as concentrated sulfuric acid.

Fluorescent dyes have typically been used for fluorescent inks for xerographic and electrographic printing of security features. However, a major drawback of fluorescent dyes is that they degrade thermally. For example, the fluorescence can be lost after about 12 days of continuous heating at 125° C. This drawback is detrimental with respect to solid ink printers because the printers need to be powered, requiring high temperature for an extended time, which has an adverse effect on the fluorescent dye.

Generally, pigments are considered the better alternative because of their improved chemical, light fastness, and thermal stability. They are also preferred by the industry because there is limited or no migration or bleeding of the colorant compound, which more easily occurs with dyes. Pigments may also be significantly less expensive than dyes, and so are attractive colorants for use in printing inks.

To overcome the problems associated with fluorescent dyes described above, the security printing industry uses hard, robust pigments containing the dye of interest. These daylight fluorescent pigments are made of a hard cross-linked polymer matrix incorporating fluorescent dyes, and are dispersed in the marking vehicle, typically liquid inks. In the hard pigment particle, the dye is isolated from interaction with other materials present in the ink and as a result, chemical degradation by the environment is prevented. In addition, mobility of the dye is severely restricted by the hard polymer matrix, which is required for any thermal degradation process.

However, these hard pigment particles also present drawbacks. For example, the size of commercially available daylight fluorescent pigments is about 3-5 microns and even higher. Currently, inks based on fluorescent pigments are being printed by rotogravure, flexographic, silk-screening, and off-set systems. Given their large size, these pigments cannot be used in ink jet printing, such as ink jet printing using solid inks or UV curable inks, because the pigments would produce physical clogging of the ink jet nozzles. For example, when preparing solid ink compositions by adding a conventional pigment to a solid ink base, pigments having a size of about 1 micron cannot be used because of their tendency to plug the nozzles of the ink jet printer, due to their large size.

Hence, the inventors worked to determine if electrochemically formed dendritic patterns could be made to fluoresce. This variant of the dendritic identifier technology may be useful for compliance, counterfeit, and cybersecurity operations, among others. In this scheme, silver dendrites were selectively coated with a fluorophore, a material that will glow brightly when illuminated with ultraviolet light, so that the dendritic structure and the information it represents only become visible when the tag is scanned using a “black” light. This may allow covert marking of items, much like what is currently done with currency and government issued identity cards which have features that are only visible under UV illumination.

### Growth of Ultra-Thin Dendritic Structures

The first development task involved the growth of virtually invisible dendrites that could be overlooked by a casual observer under ambient illumination. These were to be thin (less than about 10 nm average thickness) Ag electrodeposits on thin solid electrolyte films on suitable substrates.

The choice of electrolyte material in this exemplary embodiment, Ag-doped Ge₃Se₇, was based on the inventors' work in forming continuous 10 nm thick dendrites with this solid electrolyte. In certain embodiments, the electrolyte may be as thin as about 10 nm thick, but a thicker film was used in this embodiment to avoid difficulty in fully dissolving Ag into the base glass.

To fabricate the solid electrolyte, a 60 nm thick layer of Ge₃Se₇ base glass was deposited on a cleaned Si wafer and then coated with a 20 nm thick layer of Ag (3:1 Ge₃Se₇ to Ag ratio) without breaking vacuum in a Cressington 308 evaporation system. The Ag was then photodissolved into the base glass using UV light (λ=436 nm) in an Oriel aligner for 1,800 seconds at 4 mW/cm². These conditions ensured that the Ag was fully dissolved into the Ge₃Se₇, although other suitable conditions will be apparent to one of skill in the art.

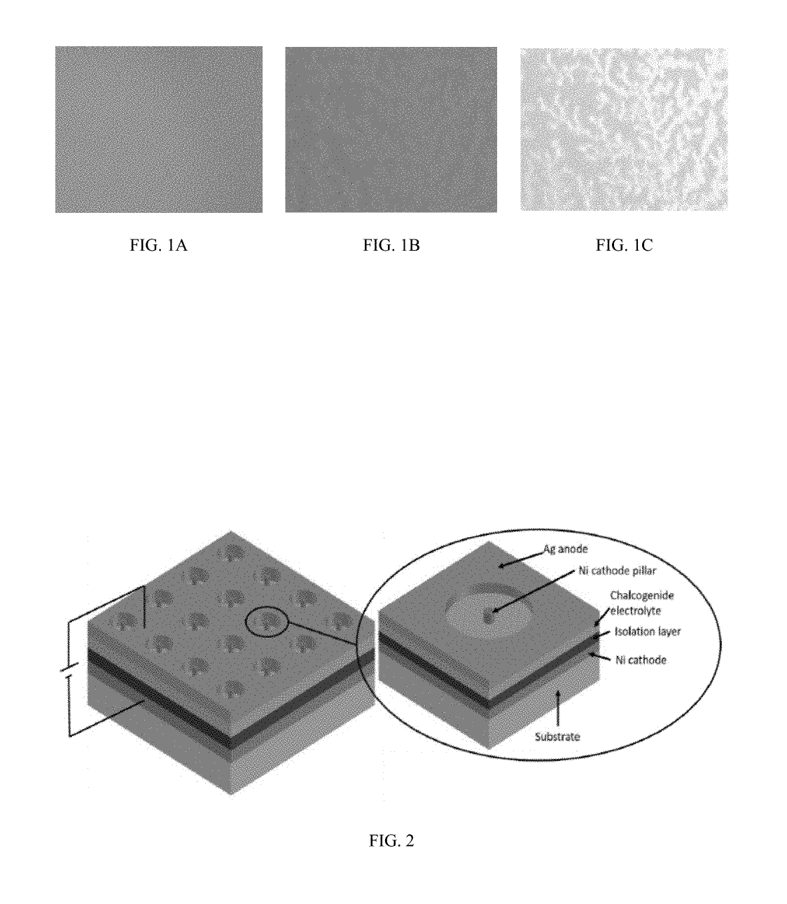

A 60 nm thick Ag film was then evaporated on the electrolyte through a shadow mask to create large rectangular anode pads. The electrolyte-coated substrate was then placed on a probe station with fine W probes connected to an Agilent 4155C semiconductor parameter. One of the probes was designated as the cathode and placed directly on the surface of the electrolyte and the other was placed in contact with a Ag anode pad. The cathode was placed approximately 1 cm from the anode and 3 V was applied. This relatively low field (3 V cm⁻¹) resulted in the slow (over 20 seconds) formation of a transparent electrodeposit, approximately 10 nm thick, as shown in FIG. 1A.

A somewhat more quantifiable result for transparency for dendrites grown under similar conditions on glass slides was obtained using an Ocean Optics Model DS200 double channel spectrometer; in this case the transmission loss due to the dendrites was less than 2% over the visible range. Indeed, as FIG. 1A shows, even at the mm scale, no discernable features are present, but a 10× magnification of the region reveals that there are indeed metallic dendrites present, albeit very faint (FIG. 1B). The micrograph in FIG. 1B was contrast enhanced to better image the dendritic electrodeposit (FIG. 1C). This experiment showed that it was indeed possible to form dendritic electrodeposits that were essentially invisible to the naked eye and were therefore covert in nature, as desired.

Even though the end result was positive, consistency of dendrite growth could be improved. The contact between the cathode probe and the electrolyte was somewhat unreliable as the electrolyte would readily deform when touched by the W probe and the contact area/resistance would vary greatly. The positioning of the probes was also inconstant and this led to different electric field and therefore different dendrite morphologies and fractal dimensions. Thus, a dendrite growth “test chip” was designed and fabricated, which had arrays of fixed cathodes coaxially aligned using precision lithographic techniques with circular anodes.

A schematic of the chip is shown in FIG. 2. Each dendrite growth location includes a Ni pillar which penetrates a layer of solid electrolyte and acts as the cathode for growth; the pillar takes the place of the probe in the growth scheme used above. All Ni pillars/cathodes are connected together by an underlying sheet of Ni which is covered in a layer of SiO₂ and then the solid electrolyte. Access to this common cathode connection is via probe pad holes in the oxide and electrolyte layers at the corners of each group of growth regions. Each cathode pillar is surrounded by a concentric Ag anode and all anodes are connected together in the same Ag layer.

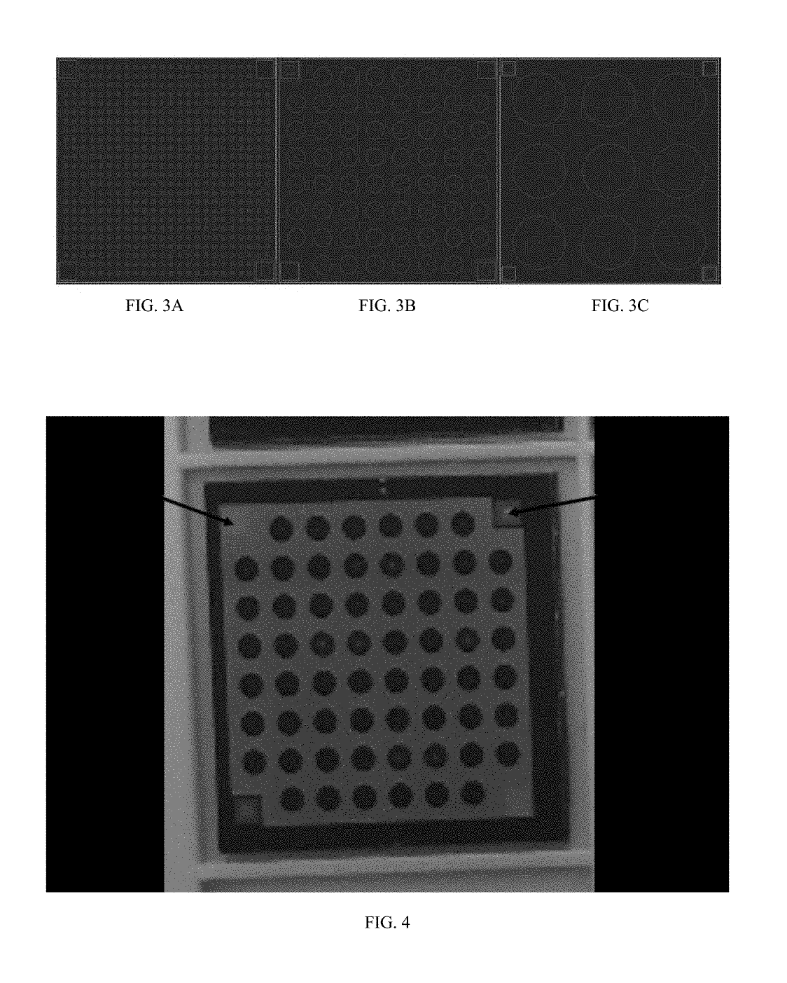

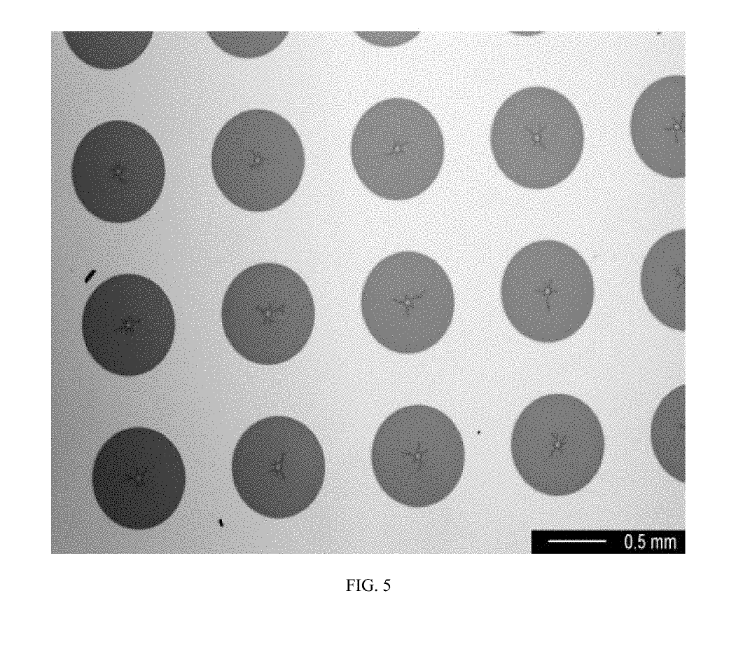

A voltage applied between cathode and anode simultaneously grows dendrites at all growth locations radially outward from each cathode, and the underlying layer of SiO₂ prevents vertical electrodeposit formation through the electrolyte between anode and cathode layers. Three growth device types were designed with different anode diameters, all with a 50 μm diameter cathode, and arranged into 1 cm by 1 cm arrays as shown in FIGS. 3A-C. Type A devices had a 200 μm diameter anode (560 devices/array); Type B had 800 μm anodes (60 devices/array), and Type C had 2400 μm anodes (9 devices/array). Multiple arrays of each device type were fabricated on 100 mm diameter silicon wafers, and each 1 cm² array was cut from the wafer for testing using a dicing saw. A photograph of a Type B array cut from a wafer is shown in FIG. 4.

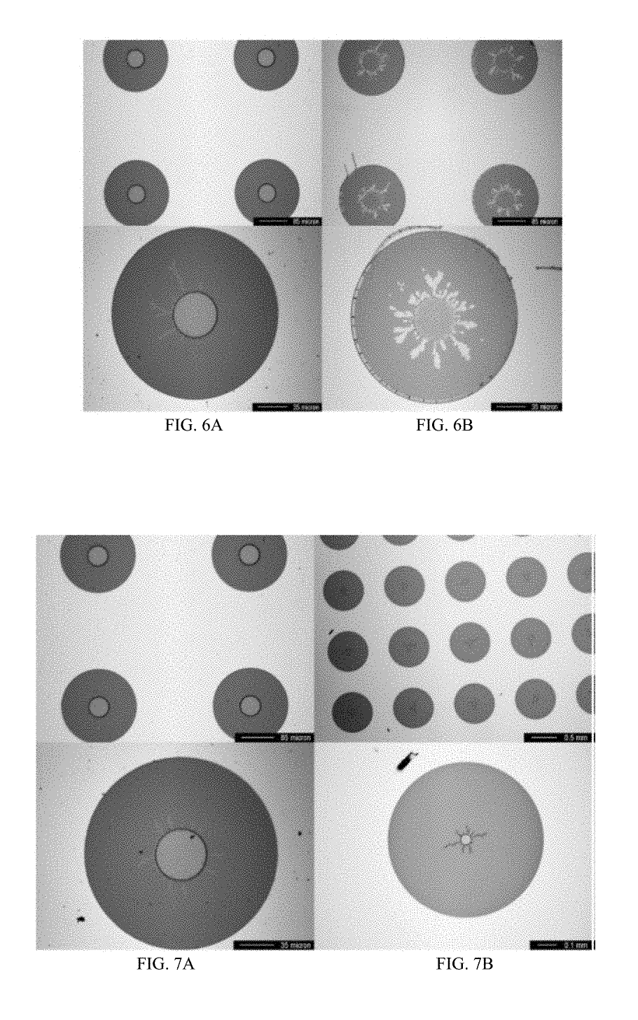

Dendrite growth was performed using an Agilent 4255C semiconductor parameter analyzer which supplied a constant voltage between cathode and anode. An example of simultaneous growth at 2 V on multiple sites is shown for a Type B sample in FIG. 5. As desired, higher voltage led to dendrites with lower fractal dimension, as the drift to diffusion ratio were higher and as illustrated in FIGS. 6A-B for Type A devices. In this case, 2 V (about 267 V cm⁻¹ initial field) led to a fractal dimension of 1.20 (FIG. 6A), whereas 1 V (about 133 V cm⁻¹ initial field) gave a dendrite with fractal dimension 1.43 (FIG. 6B).

The device size significantly impacts the dendrite morphology for the same growth voltage as the field will be higher in smaller devices and hence the drift/diffusion ratio will be higher than in larger devices. FIGS. 7A-B shows dendrites grown at 2 V in Type A and B devices. These dendrites are fairly similar in nature with a similar fractal dimension (about 1.2), despite the lower field in the Type B case, although the larger dendrite trunk spacing in the Type B case may indicate more competition for the ion supply for the larger anode-cathode spacing.

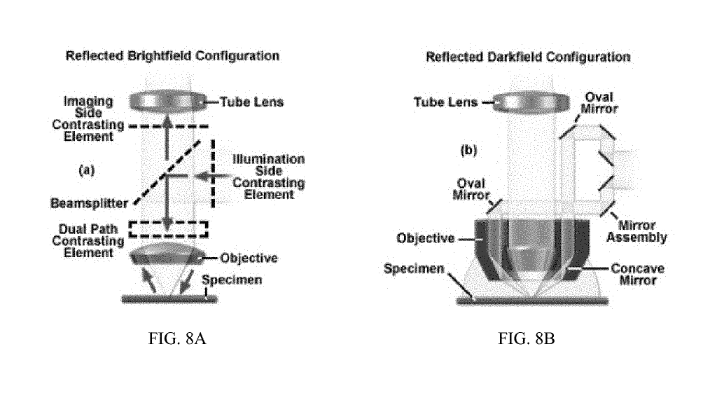

All the images in FIGS. 5, 6, and 7 were acquired using bright field imaging; these are essentially 2D projections of the dendrites, whereas the dendrites are actually 3D structures. To reveal the 3D morphology, dark field imaging was also used. FIGS. 8A-B shows the light path of both bright and dark field configurations. In the bright field configuration, all the light reflected by the sample was collected by the objective, while in the dark field configuration, only the light scattered by the sample could be collected by the objective, which means that only the relief were imaged.

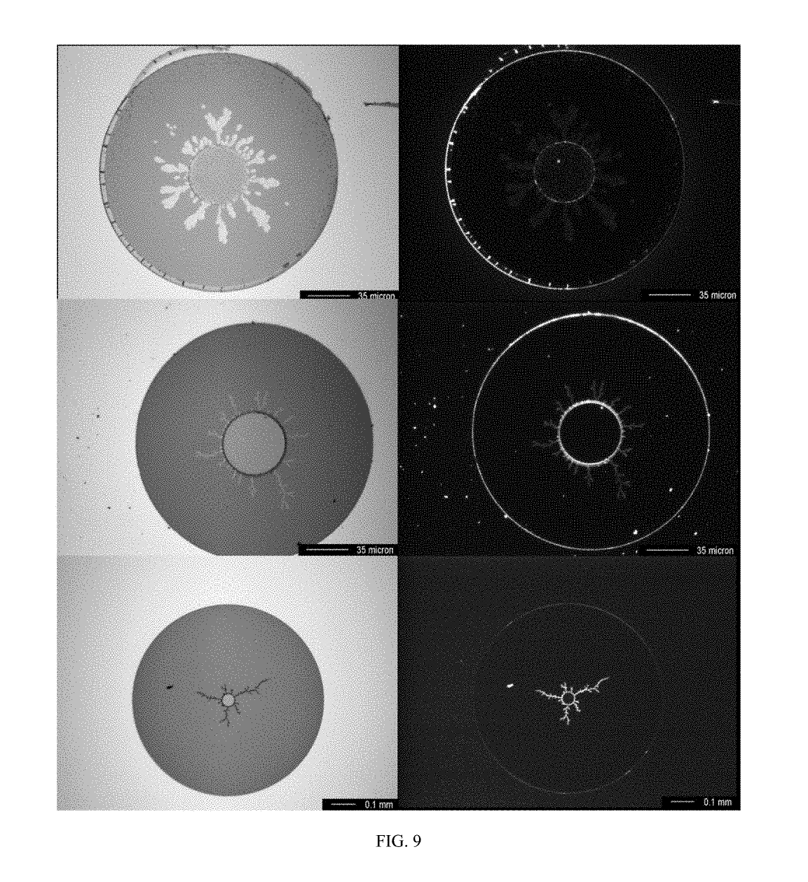

FIG. 9 shows a comparison between bright field and dark field images for dendrites in a Type A device grown with 1 V, a Type A device grown with 2 V, and a Type B device grown with 2 V. In the Type A/1 V case, the “flatter” dendrite has a strong image in light field but not in dark field. The Type A/2 V dendrite has similar contrast in both light and dark field imaging. The Type B/2 V dendrite appears dark in light field but is strongly reflecting in dark field, indicating significant relief.

The purpose of the dendrite growth test chip was to allow the growth of structures with minimal experimental variability, so that it could be ensured that the feature randomness was due to entropic growth processes and not the environment. The randomness of the growth was assessed on Type B devices which have 60 growth sites per die. A sample of 23 of these growth sites was selected and the dendrite images for growth at 2 V was captured with bright field microscopy using the same magnification, brightness, contrast, and ISO settings.

All the dendrites looked superficially similar but were indeed different from each other. Following capture, the images were converted to binary images using the Matlab image process toolbox, since the fractal analysis “Fraclac” plugin for the ImageJ suite requires this for accurate determination of the fractal dimension (FD). Threshold color filtering was applied first to clear everything except the dendrites and then the binary conversion process was applied. In order to reduce the influence of the binary process, the same image processing parameters were used for all 23 images.

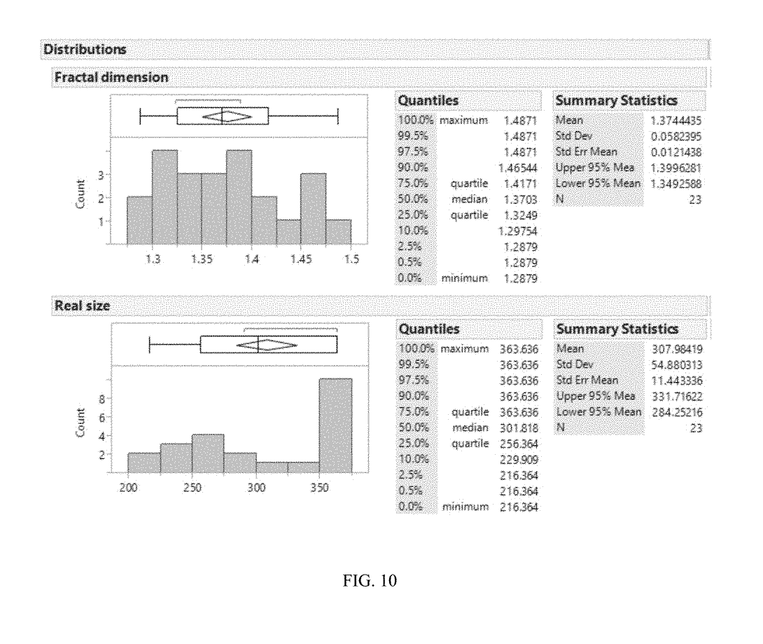

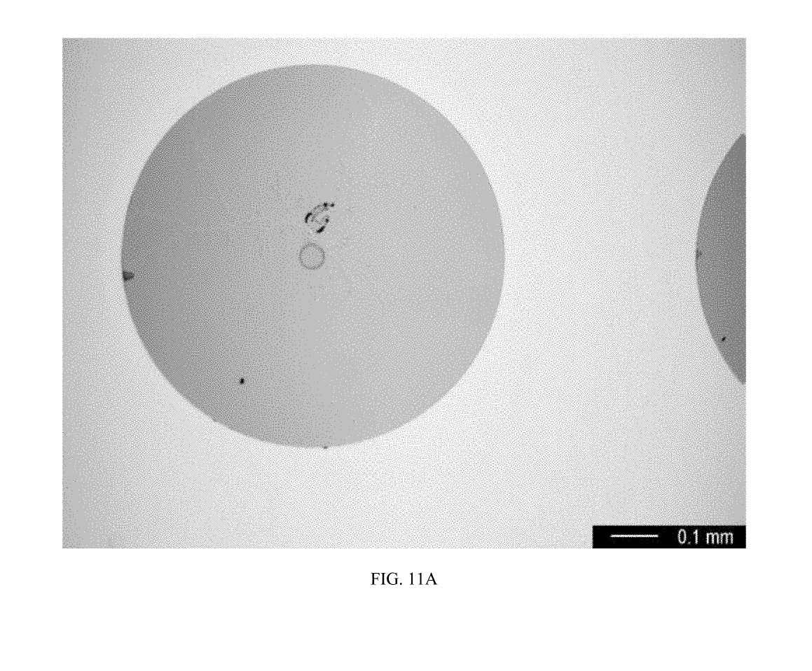

The FDs of the dendrites were analyzed using FracLac in ImageJ, and the results are summarized in FIG. 10. The FDs were distributed between 1.28 and 1.49 with an average of 1.37. The sizes of the dendrites were also measured, defined as the distance between the tip of the longest trunk and the center of the cathode. These results are also shown in FIG. 10. About 40% of the dendrites are longer than 350 μm which is very close to the anode (400 μm), while the rest of are between 200 and 350 μm, with an average size of 308 μm.

### Selective Coating of Dendrites

The second development task was to selectively coat the dendrites in a fluorophore material so that only the dendrite would fluoresce and thereby become visible under UV illumination.

Originally, a thiol-terminated UV fluorescent material, e.g., phenyl-phenanthridine with succinimide thiolate, was used that could preferentially adhere to the Ag dendrite via the thiol bond but not the surrounding material. However, in these tests there was not sufficient Ag in the electrolyte after growth to promote attachment of the dye on the surface of the electrolyte as well as on the electrodeposit. This meant that the entire surface glowed under UV illumination and the dendritic pattern was not distinct.

Instead, particle-based fluorescent inks were used, comprising less than 1 μm diameter particles of a fluorescent agent suspended in a water base, although other solvents may be suitable. The inks are widely available and are much less expensive than fluorophores. Without wanting to be limited by theory, the concept here is that the nanoscale fluorescent particles would adhere to the rough surface of the dendrite via van der Waals attraction, but would also be readily washed off of the much smoother electrolyte surface. The inks themselves appeared “milky white” under illumination with visible light but would glow with a color that depended on their fluorescent chemical used under UV illumination. The inks were applied to dendrites grown on a Type B chip and the results of this experiment are shown below.

### Fluorescence Characterization

The third development task was to characterize the fluorescence of the coated dendrites to ensure that they were not only visible under UV illumination, but that the pattern fidelity was also maintained.

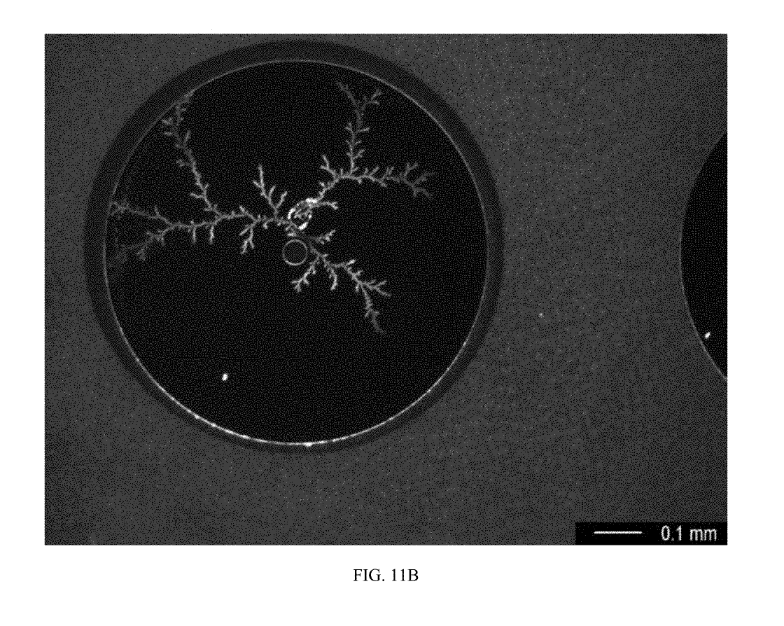

FIGS. 11A-B show the light field and dark field images of a dendritic pattern grown on a Type B site at 2 V and subsequently washed with a green fluorescent ink that appears white to the eye under room lighting. The light field pattern is barely visible at this magnification (size bar is 0.1 mm) which means that the dendrite is essentially be invisible to the naked eye. The dark field image shows that there is indeed a strong dendritic pattern present, right out to the edge of the growth region.

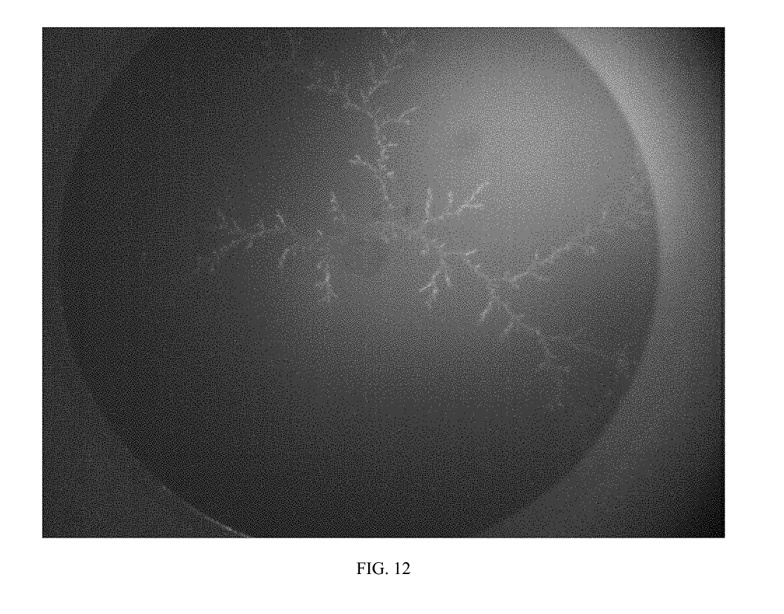

FIG. 12 shows the same ink-coated dendrite imaged using a fluorescence microscope which uses UV illumination. Here, the dendrite is much more evident, displaying greater contrast than in the visible light case of FIG. 11A.

In summary, ultra-thin dendrites were grown that are essentially invisible to the naked eye under room light; the inventors' dendrite growth chip approach eliminates experimental variables and thereby leads to high quality dendrites that can be used for statistical analyses of the patterns; and standard inexpensive UV fluorescent inks may be used to “decorate” otherwise invisible dendrites so that they become evident under UV illumination.

Various features and advantages of the invention are set forth in the following claims.

What is claimed is:

1. A method of detecting a covert marking, the method comprising:
applying a fluorescent ink to a dendritic structure to generate an ink-coated dendrite; and
imaging the ink-coated dendrite with a device to visualize the ink-coated dendrite.

2. The method of claim 1, wherein the fluorescent ink is particle-based.

3. The method of claim 2, wherein the fluorescent ink comprises particles less than 1 μm in diameter.

4. The method of claim 2, wherein the particles adhere to the dendritic structure via van der Waals attraction.

5. The method of claim 1, wherein the fluorescent ink is dye-based.

6. The method of claim 1, wherein the device is a fluorescent microscope.

7. The method of claim 1, wherein the dendritic structure comprises silver or copper.

8. The method of claim 1, wherein the dendritic structure has a thickness less than or equal to about 20 nm.

9. The method of claim 1, wherein the dendritic structure has a fractal dimension between about 1.20 and about 1.49.

10. A method of detecting a covert marking on an object, the method comprising:
applying UV light to an object including a dendritic structure; and
illuminating the dendritic structure with the UV light.

11. The method of claim 10, wherein the dendritic structure is coated with a particle-based fluorescent ink.

12. The method of claim 10, wherein the dendritic structure is coated with a dye-based fluorescent ink.

13. A dendritic structure comprising:
a unique metallic structure at least partially coated with a fluorescent ink that is not visible to the naked eye in ambient light and is visible to the naked eye when exposed to UV light.

14. The dendritic structure of claim 13, wherein the fluorescent ink is a particle-based fluorescent ink.

15. The dendritic structure of claim 14, wherein the particle-based fluorescent ink comprises particles of a fluorescent agent having a diameter less than 1 μm.

16. The dendritic structure of claim 14, wherein the particles adhere to the dendritic structure via van der Waals attraction.

17. The dendritic structure of claim 13, wherein the fluorescent ink is a dye-based fluorescent ink.

18. The dendritic structure of claim 13, wherein the unique metallic structure comprises silver or copper.

19. The dendritic structure of claim 13, wherein the dendritic structure has a thickness less than or equal to about 20 nm.

20. The dendritic structure of claim 13, wherein the unique metallic structure is a dendrite having a fractal dimension between about 1.20 and about 1.49.
